(19) United States
(12) Patent Application Publication
(10) Pub. No.: US 2017/0014443 A1
(43) Pub. Date: Jan. 19, 2017

(54) Compositions and methods for treating gastrointestinal disorders for humans and animal husbandry

(71) Applicant: Manuel Serrano, REDWOOD, CA (US)

(72) Inventor: Manuel Serrano, REDWOOD, CA (US)

(21) Appl. No.: 15/213,397

(22) Filed: Jul. 19, 2016

Related U.S. Application Data
(60) Provisional application No. 62/194,277, filed on Jul. 19, 2015.

(51) Int. Cl. A61K 31/715; A61K 38/16; A61K 36/185; A61K 36/15; A61K 31/194; A23L 33/105; A61K 31/05; A61K 31/353; A61K 31/352; A23L 33/10; A23L 33/21; A61K 36/48; A61K 9/00

(52) U.S. Cl. CPC A61K 31/715; A61K 36/48; A61K 38/168; A61K 36/185; A61K 36/15; A61K 31/194; A61K 9/0053; A61K 31/05; A61K 31/353; A61K 31/352; A23L 33/10; A23L 33/21; A23L 33/105; A23V 2002/00

(57) ABSTRACT

Compositions containing hemicelluloses, citric acid and glucose syrup, fructose or sucrose in combination with polyphenols and prebiotics, methods of preparing the compositions, and methods of treating humans or animals with the composition are provided. Also provided is a method for increasing growth rate, improving feed efficiency and decreasing scour after weaning in an animal by administering an effective amount of the composition to the animal. The prebiotics preferably are not consumed by human alimentary enzymes or harmful bacteria, such as putrefactive or pathogenic bacteria, and are consumed by beneficial bacteria, such as bifidobacteria, in the gastrointestinal tract. The polyphenols preferably decrease the amount of harmful bacteria in the gastrointestinal tract. The compositions can optionally contain a carrier or be used as a feed addition and are administered to humans or other animals in an amount sufficient to treat the gastrointestinal disorder.

The present application claims priority from the provisional application Ser. No. 62/194,277, filing date Jul. 19, 2015, entitled “Compositions and methods for treating gastrointestinal disorders for humans and animal husbandry”, hereby incorporated by reference in its entirety.

### BACKGROUND OF THE INVENTION

Several gastrointestinal disorders, including diarrhea, can be caused by an imbalance in the normal gut flora, usually an increase in harmful bacteria, including pathogenic and putrefactive bacteria such as Clostridium and Bacteroideceae, and/or a decrease in beneficial, acid forming bacteria such as bifidobacteria. Antibiotics have been used to treat diarrhea. A major drawback of using antibiotics is that they can be nonselective, killing both the harmful bacteria and the beneficial bacteria.

Diarrhea is also treated with drugs such as Loperamide HCl, commonly sold under the name Imodium™, and codeine, that act on the smooth muscle in the walls of the gastrointestinal tract to inhibit peristalsis, the rhythmic waves of muscular contraction that move the contents of the gastrointestinal tubes. These drugs are effective to treat the symptoms of diarrhea, which typically include increased force and rate of peristalsis. A major limitation, however, is that the cause of the diarrhea is not treated.

Another approach to treating gastrointestinal disorders involves coating the gastrointestinal tract with a composition containing bismuth salicylate, for example, Pepto-Bismol™. The limitation with this method of treatment is that bismuth salicylate is not always very effective at treating the symptoms of diarrhea, and does not treat the cause of diarrhea.

Gastrointestinal disorders have also been treated with dietary fiber. Dietary fiber is a general term covering a number of substances, including cellulose, hemicellulose, oligosaccharides, pectins, gums, waxes, and lignin. A more general definition is “endogenous components of plant materials in the diet that are resistant to digestion by human (intestinal) enzymes, i.e., mainly non-starch polysaccharides and lignin.” Grant-Thompson, W., “The Fiber Story,” in Gut Reactions, Understanding Symptoms in the Digestive Tract, Plenum Press, N.Y., pp. 59 (1989). Dietary fiber can be either soluble or insoluble.

Dietary fiber resists hydrolysis by human alimentary enzymes, but can be fermented by colonic microflora. In general, soluble fiber is more readily fermented than insoluble fiber. The main physiological effects of these substances are on gastric emptying and colonic transit time, and can result in improved glucose tolerance and decreased starch digestion. The fermentation of dietary fiber results in increased bacterial biomass, increased fecal mass, lowering of intracolonic pH due to production of short chain fatty acids, and production of various gases as metabolic end products. One limitation of using dietary fiber is that it can decrease the absorption of vitamins in certain individuals. Another limitation to using dietary fiber, generally, is that certain dietary fibers are fermented by both harmful and beneficial bacteria. For example, lactulose is used clinically to enrich intestinal sugar sources, since lactulose is notdigested or absorbed in human intestines, and reaches the ileum intact. While lactulose is digested by bifidobacteria, it is also digested by other intestinal bacteria, such as Escherichia Coli, and sometimes causes diarrhea, YaWaZa, K., andTamura, Z., Bifidobacteria Microflora, 1(1):39-44 (1982).

Other examples of dietary fiber that is digested by both beneficial and harmful bacteria are described in Yamada, H., et al., Cereal Foods World, 38(7):490-492, 491 (1993). Some dietary fibers can be selectively fermented by bifidobacteria, a beneficial bacteria that produces acetic and lactic acid from sugar. An example of this type of dietary fiber is wheat bran hemicellulose, which is composed mainly of arabinoxylans, Yamada, H., et al., Cereal Foods World, 38(7):490-492 (1993). Wheat bran hemicellulose apparently also suppresses the proliferation of harmful bacteria, such as Escherichia Coli. The acid produced by the bifidobacteria suppresses the adsorption of ammonia and amines produced by putrefactive bacteria such as Clostridium. Inulin and fructose oligosaccharides have been shown to have bifidogenic factors, but it is unclear why these oligosaccharides are primarily fermented by bifidobacteria. Roberfroid, M., Critical Reviews in Food Science and Nutrition, 33(2);103-148 (1993). Transglycosylated disaccharides have also been shown to increase the amount of fecal bifidobacteria and lactobacilli, and decrease the amount of Bacteroidacea and Candida spp. in the feces, Ito, M., et al., J. Nutr. Sci. Vitaminol, 39:279-288 (1993).

Certain compounds can be useful to treat gastrointestinal disorders because they selectively eliminate harmful bacteria. Some polyphenols have been reported to be useful for this purpose. Certain plants containing polyphenols have been used to treat gastrointestinal disorders,. Baldi, A., et al., Planta Medica, 58, Supplemental Issue 1, pp. A691 (1992). Polyphenols (especially flavonoids, for example, compounds with a phenyl-C3-phenyl structure, wherein the phenyl rings are functionalized with one or more hydroxygroups) derived from green tea have been reported to significantly decrease the amount of clostridium perfrigensand other clostridium spp. (putrefactive bacteria), and significantly increase the amount of bifidobacterium spp. (acid forming bacteria) in human feces, Okubo, T., et al., Biosci. Biotech. Biochem., 56(4):588-591 (1992).

It is therefore an object of the present invention to provide compositions and methods for treating gastrointestinal disorders in humans and animals.

It is a further object of the present invention to provide an inexpensive feed additive that aids digestion and/or prevents gastrointestinal disorders.

It is yet a further object of the present invention to provide a method for preparing a feed additive composition containing a prebiotic of gum Arabic or gum Ghatti, a polyphenols of punicalagins, gallotannins, epigallocatechin gallate (EGCG), or epigallocatechin (EGC), and optional a citric acid.

### SUMMARY OF THE DESCRIPTION

Compositions and methods for treating gastrointestinal disorders, including diarrhea, in humans and animals are described. The compositions include, but not limited to a prebiotics in coordination with a polyphenol, citric acid and glucose syrup, fructose or sucrose. It is believed that the composition acts by increasing the amount of beneficial bacteria, such as bifidobacteria, and reducing the amount of putrefactive and pathogenic bacteria, such as Clostridium.

In some embodiments, the prebiotic can be one or more of gum Arabic or gum ghatti. The polyphenols can be punicalagins, gallotannins, epigallocatechin gallate (EGCG), or epigallocatechin (EGC). The ratio by weight of prebiotic/polyphenols can be between 20 and 3.

The composition preferably is administered to animals as a food additive, at a dosage level of between 0.1 to 5% by weight of feed, preferably between 0.1 and 2% by weight of feed. Preferably, the composition is administered to humans as a liquid or powder added to foodstuff formulations or to drinks.

### DETAILED DESCRIPTION OF THE INVENTION

In some embodiments, the present invention discloses compositions, and methods using the compositions, for treating gastrointestinal disorders in human and animals. The compositions can treat the causes of the gastrointestinal disorders, for example, by restoring the balance of the gut flora. The gastrointestinal disorders can be caused by an imbalance of harmful bacteria and beneficial bacteria in the gastrointestinal tract, e.g., having an increased number of harmful bacteria. The present compositions can reduce the number of harmful bacteria, e.g., using a polyphenol, selectively feeding the beneficial bacteria, e.g., using a prebiotic, and creating an environment favorable to the beneficial bacteria, e.g., using an acid.

In some embodiments, the compositions can include a polyphenol that can selectively reduce harmful bacteria in the gastrointestinal tract. For example, the polyphenol can have a stronger detrimental effect on the harmful bacteria than on the beneficial bacteria, thus can selectively lower the number of harmful bacteria, e.g., reducing the ratio of harmful bacteria over the beneficial bacteria. The selective treatment of the polyphenol can slowly restore the balance of bacteria in the gastrointestinal tract, e.g., bring the ratio of harmful bacteria and beneficial bacteria to a healthy number.

In some embodiments, the polyphenol can include punicalagins, gallotannins, epigallocatechin gallate (EGCG), epigallocatechin (EGC), or any combination of these polyphenols. Other polyphenols can be used.

In some embodiments, the compositions can include a prebiotic that can selectively increase beneficial bacteria in the gastrointestinal tract. For example, the prebiotic can be fermented by one or more beneficial bacteria in the gastrointestinal tract, and thus can stimulate their growth and activities. The selective growth of beneficial bacteria can reduce the ratio of harmful bacteria over the beneficial bacteria, assisting in restoring the balance of bacteria in the gastrointestinal tract. The prebiotic can be used in conjunction with the polyphenol, to quickly balance the bacteria in the gut flora. While the polyphenol reduces the harmful bacteria, the prebiotic increases the beneficial bacteria.

In some embodiments, the prebiotic can be selected to increase the number or activity of bifidobacteria. The prebiotic can include gum Arabic and gum ghatti. Other prebiotics can be used.

In some embodiments, a ratio of the prebiotic to polyphenol can be between 20 and 3.

In some embodiments, the compositions can include an acid, which can condition the gastrointestinal tract to form an environment more favorable to the beneficial bacteria. The harmful bacteria can produce ammonia and amines, and the acid in the compositions can neutralize the ammonia and amines. The addition of the acid can form an environment more favorable to the beneficial bacteria, and thus can encourage the growth of the beneficial bacteria.

In some embodiments, the acid can include citric acid.

In some embodiments, the present invention discloses a series of compositions, and methods using the series of compositions, for treating gastrointestinal disorders in human and animals. Each composition in the series of compositions can include a prebiotic, a polyphenol, and optionally a citric acid and other components. The compositions in the series can have an increase ratio of prebiotic and polyphenol, and/or a reduced amount of citric acid.

A cause of gastrointestinal disorders is an imbalance of harmful bacteria and beneficial bacteria, e.g., there is a high number of harmful bacteria and a lower number of beneficial bacteria as compared to a healthy gut flora. Different components of the present compositions can have different effects on the bacteria, and thus different ratios of these components can be used to optimize a treatment of the gastrointestinal disorders.

In some embodiments, a high number of polyphenol can be used first, to reduce the amount of harmful bacteria, e.g., a composition having a high ratio of polyphenol and prebiotic can be used first to treat the gastrointestinal disorders. Afterward, high numbers of prebiotic can be used, to assist in increase the number of beneficial bacteria. Thus compositions having an increase ratio of prebiotic and polyphenol can be used successively, such as from 30%, to 40%, 50%, 50%, . . . to 100% of a final ratio. For example, a ratio of 5 of prebiotic/polyphenol can be used in a beginning. In the next few days, the ratio can increase, such as gradually increase, e.g., to 7, 10, 15, and 20 of prebiotic/polyphenol, for a final ratio of 20. The increase ratios can speed the recovery, since a high number of prebiotic, though selectively increasing the activities of beneficial bacteria, can also feed to the harmful bacteria.

In some embodiments, a high number of citric acid can be used first, to conditioning the gastrointestinal tract, e.g., to neutralize the ammonia and amines produced by the harmful bacteria. In times, as the amount of harmful bacteria reduces, for example, due to the effects of the polyphenol, the amount of citric acid can be reduced.

In some embodiments, a composition and method is provided for treating human gastrointestinal disorders or other disorders in which beneficial moderation of the intestinal microflora or an increasing large intestine pH. The composition can also be used to increase the growth rate and to improve feed conversion in animals and to ameliorate or cure scours or diarrhea, and also to improve and maintain general health. The composition can contain a prebiotic and a polyphenol, in combination with other optional components such as citric acid, dietary fiber, and glucose syrup, fructose or sucrose. The composition is administered orally to a human or animal in need of treatment of a gastrointestinal disorder, such as diarrhea. The composition is believed to lower the concentration of harmful bacteria, for example, putrefactive and pathogenic bacteria, and increase the concentration of beneficial bacteria, such as bifidobacteria.

As used herein, harmful bacteria are defined as those bacteria which cause gastrointestinal disorders, and include but are not limited to putrefactive and pathogenic bacteria. Putrefactive and pathogenic bacteria are defined as those bacteria that raise colonic pH by producing amines and/or ammonia, p-cresol, and indole. Types of these bacteria include but are not limited to Clostridium spp., Bacteroidaceae, and Candida spp. As used herein, beneficial bacteria are defined as those which increase the amount of small chain fatty acids, such as lactic acid, propionic acid, and acetic acid. A non-limiting example of beneficial bacteria is bifidobacteria.

A.) Prebiotics

Prebiotic can be defined as a nondigestible food ingredient that promotes the growth of beneficial microorganisms in the intestines. Prebiotics can be carbohydrates that can resist digestion in the small intestine, and reach the colon where they can be used, e.g., fermented, by intestinal bacteria as a source of energy for growth. Depending on the types of prebiotics, the prebiotics can favor or disfavor growth of specific groups of bacteria, and thus are capable of changing the composition of the bacteria in the gastrointestinal tract.

Prebiotics can selectively increase the number of specific bacterial strains, including Bifidobacteria spp, Lactobacilli and butyrate producing bacteria. For example, prebiotics can be shown to be fermented into short-chain fatty acids, which provide energy for the cells lining the gut walls. However, studies on the effectiveness of prebiotics for actual diseases are not conclusive.

In some embodiments, the prebiotic in the present compositions can be selected to increase the number or activity of bifidobacteria, including gum Arabic and gum ghatti. Other prebiotics can be used, such as chicory root, Jerusalem artichoke, dandelion greens, garlic, leeks, onion, asparagus, wheat bran, wheat flour, banana.

In some embodiments, the present compositions of a prebiotic of gum Arabic and/or gum ghatti and a polyphenol can be shown to alleviate diarrhea in a human or an animal.

In some embodiments, the compositions can be incorporated in a pharmaceutically acceptable carrier for oral consumption, such as food. The prebiotic component can be less than 10%, less than 8%, less than 5%, or less than 3% by weight of the food.

Gum Arabic can improve small intestinal absorption, and can accelerate recovery of diarrhea. Gum ghatti can improve activities of bifidobacteria, for example, by being resistant to gastrointestinal enzymes and to be degraded enzymatically only by bifidobacteria.

In some embodiments, the present compositions can include a series of compositions, in which each composition can have an increased amount of prebiotic, which can increase a ratio of prebiotic and polyphenol.

To treat a gastrointestinal disorder, a composition having a low number of prebiotic can be used first, followed by compositions having increased numbers of prebiotic, e.g., in a few days. For example, in subsequent days, the compositions can increase by 10%, then 20%, then 30%, . . . of the original number of prebiotic. Alternatively, the compositions can be 10%, then 20%, then 30%, . . . of the final number of prebiotic.

The gradual increase of the prebiotic amount can partially discourage the growth of harmful bacteria, since there can be consumption of prebiotic by the harmful bacteria. As the harmful bacteria is reduced, for example, by the treatment of polyphenol, the amount of prebiotic can increase, to encourage the growth of beneficial bacteria, assisting to restore the balance of bacteria in the gastrointestinal tract. The gradual increase of the prebiotic amount can be accompanied by a gradual reduction in an amount of polyphenol.

B.) Polyphenols

As used herein, polyphenols are defined as molecules with two or more phenol moieties. Useful polyphenols include flavonoids, such as tannins, punicalagins, gallotannins, epigallocatechin gallate (EGCG), epigallocatechin (EGC), aromadendrines, anthocyanins, catecholins, catechins and taxifolins. The polyphenol can lower the amount of harmful bacteria, such as clostridium, without lowering the amount of beneficial bacteria, such as bifidobacteria. Preferred polyphenols have a molecular weight range of between 280 and 6,000.

In some embodiments, the polyphenol in the present compositions can include punicalagins, gallotannins, epigallocatechin gallate (EGCG), and epigallocatechin (EGC). The present compositions of a prebiotic of gum Arabic and/or gum ghatti and a polyphenol of punicalagins, gallotannins, epigallocatechin gallate (EGCG), and/or epigallocatechin (EGC) can be shown to alleviate diarrhea in a human or an animal.

In some embodiments, the polyphenol component can be less than 10%, less than 8%, less than 5%, or less than 3% by weight of a pharmaceutically acceptable carrier for oral consumption, such as food.

In some embodiments, the polyphenol can include taxifolin, since it is found in the Larix tree, which also contains arabinogalactan, a prebiotic, which is a dietary fiber that beneficially nourishes the beneficial bacteria already in the large bowel or colon. A composition can be formed by a preparation of arabinogalactan containing polyphenols. In plants, arabinogalactan is a major component of gum arabic and gum ghatti.

In a typical process for preparing arabinogalactan, wood from a tree of the genus Larix, for example, Larix occidentalix Nuttall (Western Larch), is chipped or pulverized. The arabinogalactan is then extracted with warm water. Polyphenols, including taxifolens, are also extracted by this process. To prepare purified arabinogalactan, the polyphenols are removed, for example, by reacting the crude extract with MgO. However, retention of the polyphenols is desired, since both the dietary fiber and the polyphenols are useful for treating gastrointestinal disorders. The process can be optimized for maximum extraction of polyphenols by increasing the water temperature and/or by raising the pH to between 7 and 12 by adding a base such as ammonia, or sodium, calcium or potassium hydroxide.

As used herein, an arabinogalactan is defined as an oligosaccharide containing a β-(1,3)-linked galactan backbone with sideaverage molecular weight is between 3,000 and 2,500,000, and more preferably, between 3,000 and 100,000. Arabinogalactans can be derived from Larix trees. Preferably, the ratio of arabino groups to galactose groups is between 01:1 and 1:1.

In some embodiments, the present compositions can include a series of compositions, in which each composition can have a reduced amount of polyphenol, which can increase a ratio of prebiotic and polyphenol.

Since a gastrointestinal disorder can be caused by a high ratio of harmful bacteria over beneficial bacteria, a composition having a high number of polyphenol can be used first, to reduce the number of harmful bacteria. Afterward, compositions having reduced number of polyphenol can be used, e.g., in a few days, since the first polyphenol can be effective, and the amount of harmful bacteria can decrease. For example, in subsequent days, the compositions can have 90%, then 80%, then 70%, . . . of the original number of polyphenol. In some embodiments, the number of polyphenol can be reduced to zero, e.g., when the gastrointestinal disorder is cured or almost cured.

The gradual reduction of polyphenol amount can be accompanied by a gradual increase in an amount of prebiotic. The gradual increase of prebiotic can encourage the growth of beneficial bacteria, further assisting to restore the balance of bacteria in the gastrointestinal tract.

C.) Dietary Fiber.

In some embodiments, the compositions can include a dietary fiber. As used herein, dietary fiber is defined as endogenous components of plant materials in the diet that are resistant to digestion by human or other animal (intestinal) enzymes. Dietary fibers include but are not limited to cellulose, hemicellulose, oligosaccharides, pectins, gums, waxes, and lignin. The dietary fiber can be soluble or insoluble, but soluble fibers are preferred. Soluble fiber is defined as fiber that is soluble in water, and insoluble fiber is defined as a fiber that is insoluble in water. The dietary fiber can be highly branched, for example, more than one branch per 100 in-chain units. As used herein, hemicellulose is defined as a polysaccharide found in plant cell walls in association with cellulose and lignin, which is soluble in and extractable by dilute alkaline solutions. The average molecular weight ranges for hemicelluloses are between 3,000 and 2,500,000, or between 3,000 and 100,000.

Dietary fiber is not digested by human alimentary enzymes, and thus can reach the ileum and large intestine largely intact, to be digested by bacteria in the ileum and large intestine. Thus different types of dietary fiber can feed different types of bacteria.

Several dietary fibers are known to have bifidogenic factors, e.g., functioning as prebiotics. These fibers can include arabinoxylan, galactomannan, inulin, fructose oligosaccharide, transglycosylated oligosaccharides, and wheat bran hemicellulose. The dietary fibers can be used in the present compositions in combination with polyphenols.

In some embodiments, the present compositions can include a hemicellulose dietary fiber for promoting activities of bacteria in the gastrointestinal tract. The hemicellulose dietary fiber can be selected to be not digested by bacteria other than bifidobacteria, and thus function as an efficient sugar source for bifidobacteria. The hemicellulose is preferably soluble in aqueous solutions at a pH less than or equal to 8. For example, bifidobacteria can utilize hemicellulose, and thus hemicellulose has potential as a prebiotic compound.

D.) Prebiotic—Polyphenol Composition

Prebiotics and polyphenols can be combined by mixing. The ratio of fiber/polyphenol by weight in the composition is preferably between 20 and 3. This composition can optionally be combined with a carrier that is pharmaceutically acceptable for oral administration. When combined with a carrier, the weight percent of the composition/carrier is preferably between 1 and 10. Typical carriers are food and water. If soluble fiber is used, the combination of an aqueous carrier and the fiber will be a solution. If insoluble fiber is used, the combination of an aqueous carrier and the fiber will be a suspension. The compositions can include an inert diluent or an edible carrier. They may be enclosed in gelatin capsules or compressed into tablets. For the purpose of oral therapeutic administration, the composition can be incorporated with excipients and used in the form of tablets, troches, suppositories or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as fatty oil. In addition, dosage unit forms can contain various other materials which modify the physical form of the dosage unit, for example, coatings of sugar, shellac, or other enteric agents.

The composition can be administered as a component of an elixir, suspension, syrup, wafer, chewing gum or the like.

A syrup may contain, in addition to the active compounds, sucrose as a sweetening agent and certain preservatives, dyes and colorings and flavors.

In some embodiments, the composition can include an acid, such as citric acid. The acid can reduce the pH, for example, to form an environment more favorable to the beneficial bacteria. There can be an imbalance of bacteria, with a ratio of harmful bacteria over beneficial bacteria higher than normal, the environment can be more basic, e.g., higher pH. Thus the addition of an acid can bring the pH level to a more favorable level.

The acid additives can have a different effect than a buffer acid, since the buffer acid tends to regulate the pH level, while the acid tends to lower the pH value. For treatment of high pH disorder, an acid can be more effective.

In some embodiments, the present series of compositions can have a reduced amount of citric acid. In the series, a high number of citric acid can be used first, since there can be a high ratio of harmful bacteria over beneficial bacteria. The high number of citric acid can condition the gastrointestinal tract, e.g., to neutralize the high ammonia and amines produced by the harmful bacteria. After a certain times, such as a few days, the number of citric acid can be reduced, for example, gradually reduced, since the treatment can be effective and the amount of harmful bacteria reduces.

F.) Treatment of Gastrointestinal Disorders

The composition is useful to treat gastrointestinal disorders, such as diarrhea. The composition is administered to a human or animal in need of treatment thereof. Gastrointestinal disorders are well known to those in the art.

Examples of gastrointestinal disorders include but are not limited to diarrhea, distension of the abdomen, diverticulitis,constipation, and irritable bowel syndrome. Several gastrointestinal disorders are known to be caused by an increase in harmful bacteria, or a decrease in beneficial bacteria in the gastrointestinal tract. The composition is also useful to treat hepatic encephalotomy associated with cirrhosis of the liver. Typical systemic dosages for treatment of gastrointestinal disorders are those ranging from 10 mg/kg to 300 mg/kg per day as a single daily dose or divided daily doses.

The composition is administered for a sufficient time period to alleviate the undesired symptoms and the clinical signs associated with the gastrointestinal disorder being treated. The concentration of the components in the composition will depend on absorption, inactivation, and excretion rates of the components as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the claimed composition.

The composition can also be mixed with other active materials which do not impair the desired action, or with materials that supplement the desired action, such as compounds that treat the symptoms of peristalsis. Because intake of dietary fiber may adversely affect the absorption of vitamins and minerals in certain individuals, it can be desirable to combine the composition with a vitamin and/or mineral supplement.

Incorporation into Animal Feed

The dietary fiber-polyphenol composition can be added to animal feed. Animal feeds include but are not limited to poultry feed, swine feed, horse feed, feed for early-weaned calves, and dog and cat food. Typical dosage ranges are between 0.1 to 5% by weight of the animal feed, preferably between 0.1 and 2% by weight of the animal feed. By increasing the amount of beneficial bacteria, and lowering the amount of harmful bacteria, the health, feed conversion efficiency and growth rate of the animal are expected to increase. Diarrhea, especially during and after weaning, will decrease.

This invention has been described wWith reference to its preferred embodiments. Variations and modifications of the invention will be obvious to those skilled in the art from the foregoing detailed description of the invention. It is intended that all of these variations and modifications be included within the scope of the appended claims.

What is claimed is:

1. A composition comprising
a prebiotic; and
a polyphenol, wherein the polyphenol functions to selectively reduce an amount of harmful bacteria as compared to an amount of beneficial bacteria in a gastrointestinal tract.

2. A composition as in claim 1
wherein the prebiotic functions to increase a number of activities of bifidobacteria.

3. A composition as in claim 1
wherein the prebiotic comprises gum Arabic.

4. A composition as in claim 1
wherein the prebiotic comprises gum ghatti.

5. A composition as in claim 1
wherein the harmful bacteria function to raise colonic pH.

6. A composition as in claim 1
wherein the beneficial bacterial function to increase an amount of small chain fatty acids.

7. A composition as in claim 1
wherein the polyphenol comprises punicalagins, gallotannins, epigallocatechin gallate (EGCG), epigallocatechin (EGC), or any combination thereof.

8. A composition as in claim 1
wherein the polyphenol is a flavonoid.

9. A composition as in claim 1
wherein the flavonoid is selected from the group consisting of tannins, taxifolins, catecholines, anthocyanins, and catechins.

10. A composition as in claim 1 further comprising
a dietary fiber, wherein the dietary fiber is derived from a tree of the genus Larix.

11. A composition as in claim 1 further comprising
citric acid, wherein the citric acid comprises an amount configured to provide an acidic environment for the growth of the beneficial bacteria

12. A composition as in claim 1 further comprising
a pharmaceutically acceptable carrier for oral consumption.

13. A composition as in claim 1
wherein the composition is configured to be administered in a dosage of between 10 and 300 mg/kg body weight of a patient.

14. A composition as in claim 1
wherein the composition is added to food in a concentration of between 0.1 and 5% by weight of the food.

15. A composition as in claim 1
wherein the composition is added to food in a concentration of between 0.1 and 2% by weight of the food.

16. A composition as in claim 1
wherein the composition is added to food in a concentration effective to increase the relative ratio of bifidobacteria to clostridia.

17. A series of compositions,
wherein each composition comprises
a prebiotic; and
a polyphenol, wherein the polyphenol functions to selectively reduce an amount of harmful bacteria as compared to an amount of beneficial bacteria in a gastrointestinal tract,
wherein a ratio of the prebiotic and the polyphenol increases in the series of compositions.

18. A series of compositions as in claim 1
wherein the increase of the ratio of the prebiotic and the polyphenol depends on a rate of recovery of a gastrointestinal disorder.

19. A method for treating gastrointestinal disorders, the method comprising administering a series of a compositions,
wherein each composition comprises
a prebiotic; and
a polyphenol, wherein the polyphenol functions to selectively reduce an amount of harmful bacteria as compared to an amount of beneficial bacteria in a gastrointestinal tract,
wherein a ratio of the prebiotic and the polyphenol increases in the series of compositions.

20. A method as in claim 1
wherein the series of compositions is effective to alleviate diarrhea in a human or an animal.
